(19) United States
(12) Patent Application Publication
(10) Pub. No.: US 2022/0198330 A1
(43) Pub. Date: Jun. 23, 2022

(54) METHOD AND SYSTEM FOR GENERATING MOLECULAR STRUCTURE OF CHEMICAL COMPOUND

(71) Applicant: Alibaba Group Holding Limited, George Town (KY)

(72) Inventors: Qi Zhang, Hangzhou (CN); Jinquan Sun, Hangzhou (CN); Yu Wang, Hangzhou (CN)

(21) Appl. No.: 17/534,841

(22) Filed: Nov. 24, 2021

(30) Foreign Application Priority Data
Dec. 14, 2020 (CN) 202011467155.8

(51) Int. Cl. G06N 20/00

(52) U.S. Cl. CPC G06N 20/00

(57) ABSTRACT

Generation of a molecular structure of a chemical compound is disclosed. A target agent is trained based on a first reward and a second reward, the first reward being a reward determined by a model likelihood of a target neural network model, the second reward being a reward self-defined based on target requirements, and the target agent being used to determine a molecular compound structure. A target molecular structure of a chemical compound is generated using the target agent.

### CROSS REFERENCE TO OTHER APPLICATIONS

This application claims priority to People's Republic of China Patent Application No. 202011467155.8 entitled METHOD, MEANS, AND NON-VOLATILE STORAGE MEDIUM FOR GENERATING A MOLECULAR STRUCTURE OF A CHEMICAL COMPOUND filed Dec. 14, 2020 which is incorporated herein by reference for all purposes.

### FIELD OF THE INVENTION

The present application relates to the field of generating a molecular structure of a chemical compound.

### BACKGROUND OF THE INVENTION

The REINVENT technique (a technique for generating small molecule compounds) describes using a reinforcement learning technique in training agents to generate SMILES (Simplified Molecular Input Line Entry System). The log-likelihoods of pretraining model generated sequences serve as rewards to enable a training agent to generate SMILES strings having the same distribution as the training sets. Because the small molecule sequences generated by the REINVENT technique are insufficiently diverse, the DrugEx technique (another technique for generating small molecule compounds) was used to increase diversity in the agent training technique.

However, the generation techniques are generating small molecule sequences that still have the following limitations: 1. A poor fit exists between the sequence generation technique by the small molecule compound structure-generating agent and the pretraining operation. The result is exposure bias. 2. The SMILES string generation technique cannot be controlled. It is not possible to generate small molecule compounds having a required characteristic.

### BRIEF DESCRIPTION OF THE DRAWINGS

Various embodiments of the invention are disclosed in the following detailed description and the accompanying drawings.

FIG. 1 is a functional diagram illustrating a programmed computer system for generating a molecular structure of a chemical compound in accordance with some embodiments.

FIG. 2A is a flowchart of an embodiment of a process for generating a molecular structure of a chemical compound.

FIG. 2B is a flowchart of an embodiment of a process for training a target agent based on a first reward and a second reward.

FIG. 2C is a flowchart of yet another embodiment of a process for generating a molecular structure of a chemical compound.

FIG. 2D is a flowchart of yet another embodiment of a process for generating a molecular structure of a chemical compound.

FIG. 2E is a flowchart of an embodiment of a process for pretraining an initial neural network model.

FIG. 2F is a flowchart of another embodiment of a process for pretraining an initial neural network model.

FIG. 3 is a flowchart of yet another embodiment of a process for generating a molecular structure of a chemical compound.

FIG. 4 is a flowchart of yet another embodiment of a process for generating a molecular structure of a chemical compound.

FIG. 5 is a structural diagram of an embodiment of a system for generating a molecular structure of a chemical compound.

FIG. 6 is a structural diagram of another embodiment of a system for generating a molecular structure of a chemical compound.

FIG. 7 is a structural diagram of yet another embodiment of a system for generating a molecular structure of a chemical compound.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Some of the terms that will appear in describing embodiments of the present application are explained below:

Design of small molecule structures for drugs: the generation of compounds that, having a novel scaffold and being characterized by preliminary biological activity, are core structures of small molecules for drugs.

Agent: belongs to a field of artificial intelligence; a computer entity that, situated in a certain environment, is able to function continuously and autonomously while having the characteristics of being situated, reactive, social, and proactive, as well as other characteristics. The Agent can be viewed as capable of perceiving its environment through sensors and performing actions that act on that environment. The Agent can be hardware (such as a robot) or software.

Scheduled sampling: for deciding input at each operation during decoding by establishing a probability value.

Reinforcement learning: for describing and solving the problem of maximizing returns or achieving specific goals through learning strategies in the process of an agent interacting with an environment.

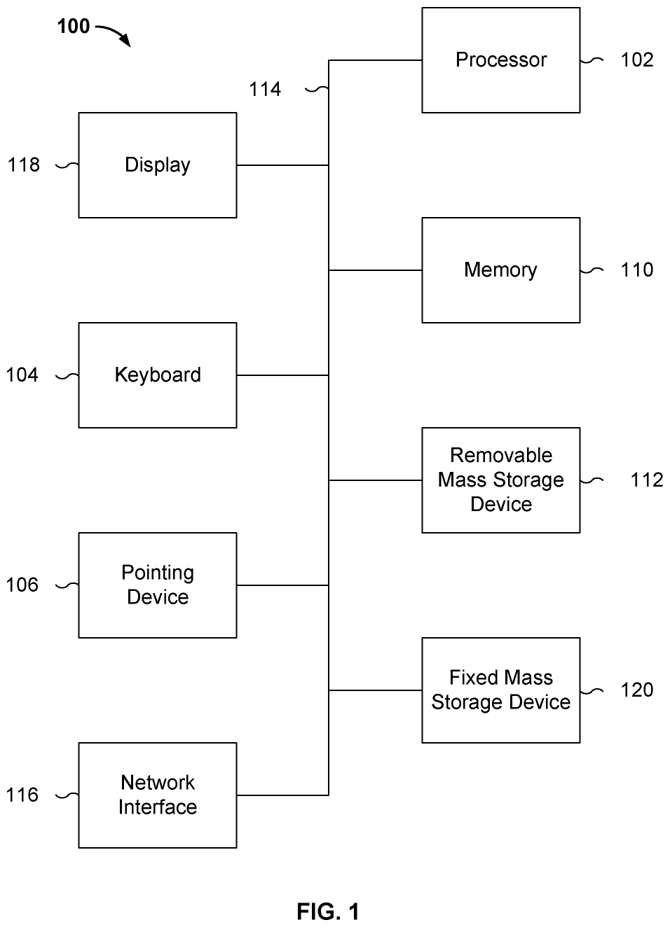

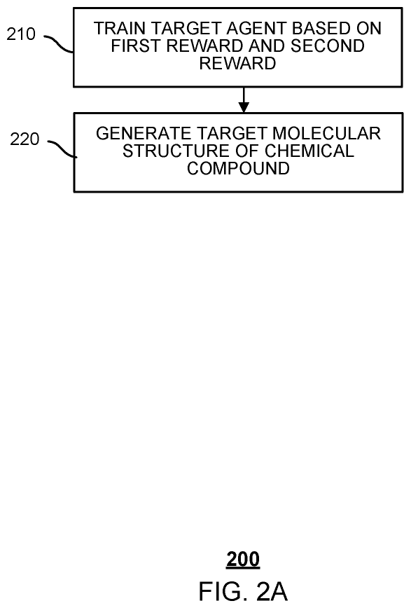

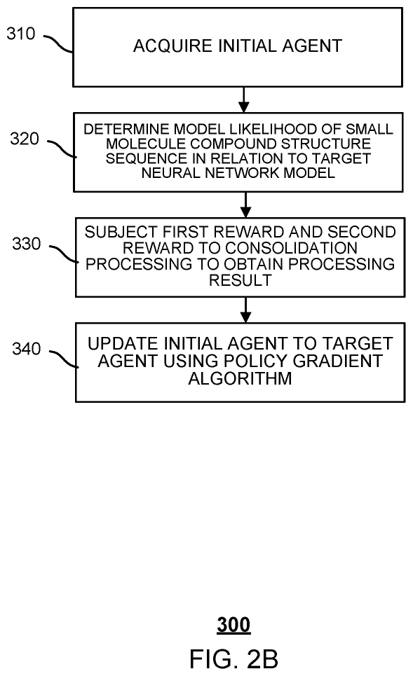

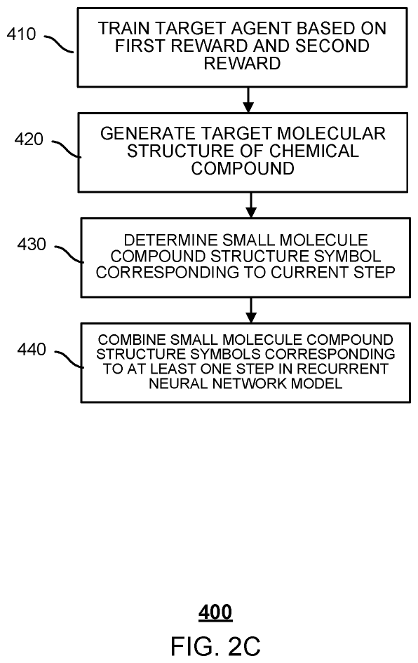

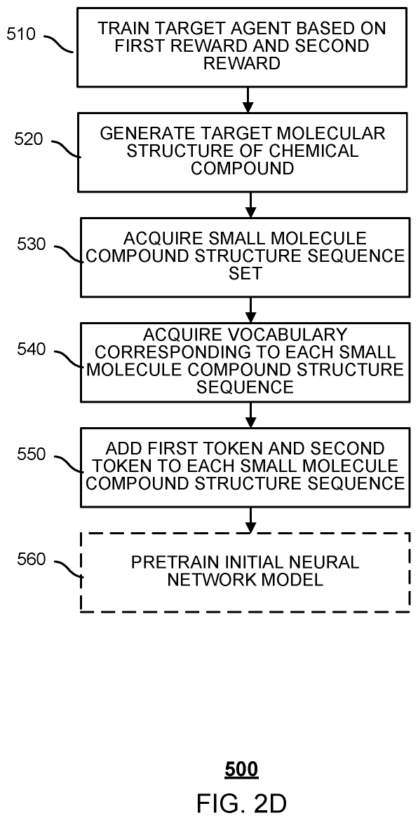

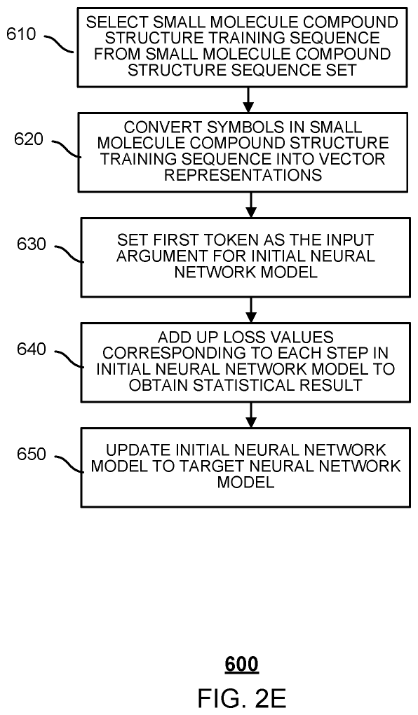

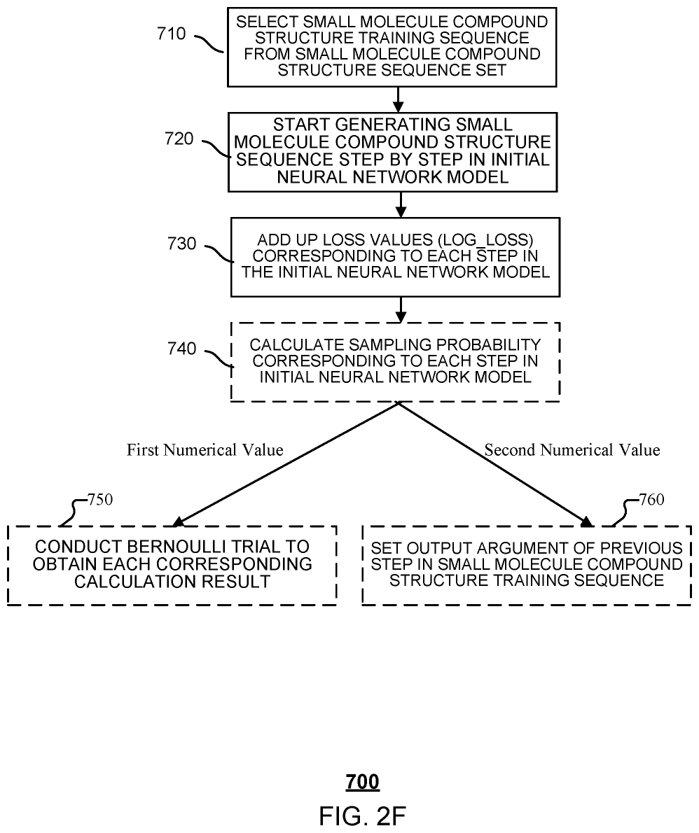

FIG. 1 is a functional diagram illustrating a programmed computer system for generating a molecular structure of a chemical compound in accordance with some embodiments. As will be apparent, other computer system architectures and configurations can be used to generate a molecular structure of a chemical compound. Computer system 100, which includes various subsystems as described below, includes at least one microprocessor subsystem (also referred to as a processor or a central processing unit (CPU)) 102. For example, processor 102 can be implemented by a single-chip processor or by multiple processors. In some embodiments, processor 102 is a general purpose digital processor that controls the operation of the computer system 100. Using instructions retrieved from memory 110, the processor 102 controls the reception and manipulation of input data, and the output and display of data on output devices (e.g., display 118). In some embodiments, processor 102 is configured to generate a molecular structure of a chemical compound with respect to FIGS. 2A-4.

Processor 102 is coupled bi-directionally with memory 110, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 102. Also as is well known in the art, primary storage typically includes basic operating instructions, program code, data and objects used by the processor 102 to perform its functions (e.g., programmed instructions). For example, memory 110 can include any suitable computer-readable storage media, described below, depending on whether, for example, data access needs to be bi-directional or uni-directional. For example, processor 102 can also directly and very rapidly retrieve and store frequently needed data in a cache memory (not shown).

A removable mass storage device 112 provides additional data storage capacity for the computer system 100, and is coupled either bi-directionally (read/write) or uni-directionally (read only) to processor 102. For example, storage 112 can also include computer-readable media such as magnetic tape, flash memory, PC-CARDS, portable mass storage devices, holographic storage devices, and other storage devices. A fixed mass storage 120 can also, for example, provide additional data storage capacity. The most common example of mass storage 120 is a hard disk drive. Mass storages 112 and 120 generally store additional programming instructions, data, and the like that typically are not in active use by the processor 102. It will be appreciated that the information retained within mass storages 112 and 120 can be incorporated, if needed, in standard fashion as part of memory 110 (e.g., RAM) as virtual memory.

In addition to providing processor 102 access to storage subsystems, bus 114 can also be used to provide access to other subsystems and devices. As shown, these can include a display monitor 118, a network interface 116, a keyboard 104, and a pointing device 106, as well as an auxiliary input/output device interface, a sound card, speakers, and other subsystems as needed. For example, the pointing device 106 can be a mouse, stylus, track ball, or tablet, and is useful for interacting with a graphical user interface.

The network interface 116 allows processor 102 to be coupled to another computer, computer network, or telecommunications network using a network connection as shown. For example, through the network interface 116, the processor 102 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 102 can be used to connect the computer system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Additional mass storage devices (not shown) can also be connected to processor 102 through network interface 116.

An auxiliary I/O device interface (not shown) can be used in conjunction with computer system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

The computer system shown in FIG. 1 is but an example of a computer system suitable for use with the various embodiments disclosed herein. Other computer systems suitable for such use can include additional or fewer subsystems. In addition, bus 114 is illustrative of any interconnection scheme serving to link the subsystems. Other computer architectures having different configurations of subsystems can also be utilized.

FIG. 2A is a flowchart of an embodiment of a process for generating a molecular structure of a chemical compound. In some embodiments, the process 200 is implemented in the computer system 100 of FIG. 1 and comprises:

In 210, the computer system trains a target agent based on a first reward and a second reward. In some embodiments, the first reward is a reward determined by a model likelihood of a target neural network model, the second reward is a reward self-defined based on target requirements, and the target agent is configured to determine a molecular compound structure. As an example, the target requirement includes that the expected generated small molecule compound structure does not contain a Cl atom.

In 220, the computer system generates, by the target agent, a target molecular structure of the chemical compound.

In some embodiments, the target agent is trained based on the first reward and the second reward. In some embodiments, the first reward is a reward determined by a model likelihood of a target neural network model, the second reward is a reward self-defined based on target requirements, and the target agent is configured to determine a molecular compound structure. In some embodiments, a target compound molecular structure is generated by the target agent.

Because a scheduled sampling technique is used in the pretraining operation, and the target agent is trained based on a first reward and a second reward, a better fit exists between the target agent-based process of generating small molecule compound structures and the pretraining operation. Because the first reward is a reward determined by the model likelihood of the target neural network model and the second reward is a reward self-defined based on target requirements, the generated small molecule compound structure can meet the specific expectation of the user regarding generation of small molecule compound structures, and the small molecule compound structure generating process can become partially controllable. Moreover, enabling the target agent to “make the most of itself” while imitating existing distributions of existing small molecule compound structures is also possible, and thus a greater number of novel small molecule compound structures can be generated.

Therefore, in this embodiment, a better fit between the target agent-based generation of small molecule compound structures and the pretraining operation exists, and generating expected small molecule compound structures can be achieved. The small molecule compound structure generation efficiency and controllability can be increased. The technical limitations of conventional techniques, namely, the poor fit between the process of agent generation of sequences and the pretraining operation, and the inability to generate small molecule compound structures that meet expectations are reduced.

In some embodiments, the process for generating a molecular structure of a chemical compound can be understood as a process for generating small molecule drug structures that is customizable and based on scheduled sampling. Unlike conventional generation techniques, the present application introduces scheduled sampling in the pretraining operation. In other words, the target agent is trained based on a first reward and a second reward, which increases the fit between the target agent-based generation of small molecule compound structures and the pretraining operation, while avoiding exposure bias.

Please note that, with the process for generating a molecular structure of a chemical compound, small molecule compound molecular structure sets for input in actual application contexts already exist. The output of the process can be a novel small molecule compound structure with the physical and chemical properties expected by the user. This process can be implemented as a small molecule drug design service provided for the user, i.e., customized generation of a novel molecular structure having the features or nature expected by the user.

FIG. 2B is a flowchart of an embodiment of a process for training a target agent based on a first reward and a second reward. In some embodiments, the process 300 is an implementation of operation 210 of FIG. 2A and comprises:

In 310, the computer system acquires an initial agent.

In 320, the computer system determines a model likelihood of a small molecule compound structure sequence, generated by an initial agent, in relation to a target neural network model as a first reward, and determines a molecular structure limiting conditions set based on the target requirements as a second reward.

In 330, the computer system subjects the first reward and the second reward to consolidation processing to obtain a processing result.

In 340, based on the processing result, the computer system updates the initial agent to a target agent using a policy gradient algorithm.

In some embodiments, the first reward is a reward determined by the model likelihood of a target neural network model, and the second reward is a reward self-defined based on the target requirements.

In some embodiments, the small molecule compound structure sequence is a sequence composed of multiple atom symbols. The hydrogen atoms are typically omitted from the small molecule compound sequence. Also, a few symbols that represent chemical bonds exist, and a few bracket symbols that represent branches exist.

In some embodiments, the initial agent is an initial computer entity, such as a smart machine or intelligent software, configured to generate molecular compound structures. In some embodiments, the target agent is the target computer entity, such as a target smart machine or target intelligent software, obtained after the initial agent is subjected to scheduled sampling and updated processing.

In some embodiments, the model likelihood of a small molecule compound structure (SMILE) sequence, generated by an initial agent, in relation to the target neural network model is defined as a first reward R1:

R₁(A)=log Πₜ₌₁^{T}πₚᵣₑₜᵣₐᵢₙ(aₜ|sₜ)

The action sequence A=a₁, a₂, . . . , a_{T} of the generation process corresponds to the generated small molecule compound (SMILES) representation; sₜ corresponds to the current cell status of the pretraining neural network model.

In some embodiments, the first reward R1 is determined by adopting a scheduled sampling technique, with the result that SMILE strings generated by the initial agent are more sufficient and have a higher likelihood on the pretraining network, i.e., have a distribution that better fits the existing small molecule compound structure (SMILE) sequence set. At the same time, since the pretraining operation uses the scheduled sampling technique, the target agent “makes the most of itself” while imitating existing distributions and thus generates a greater number of novel small molecule compound structures.

In some embodiments, defining the second reward R2 for SMILE strings generated with the molecular structure limiting conditions set based on specific target requirements is possible. For example, in the event that the expected generated small molecule compound structure does not contain a Cl atom, the second reward R2 is defined as follows:

$${R}_{2} ( A ) = \{ \begin{matrix}1 If the generated result does not contain a Cl atom , and the molecular structure is valid \\ 0 If the generated structure is invalid \\ - 1 If the generated molecular structure contains a Cl atom\end{matrix}$$

In the event that R₂(A)=1 then the expected generated small molecule compound structure does not contain a Cl atom, and the molecular structure is valid.

In the event that R₂(A)=0, it means that the expected generated small molecule compound structure is invalid, and if R₂(A) is to become equal to 1, the expected generated small molecule compound structure needs to be valid.

In the event that R₂(A)=−1, it means that the expected generated small molecule compound structure contains a Cl atom, and if R₂(A) is to become equal to 1, the expected generated small molecule compound structure cannot contain a Cl atom.

In some embodiments, the second reward is determined from molecular structure limiting conditions set based on target requirements. In other words, the second reward is determined in a customized manner, which can make the small molecule structure generating process partially controllable and can achieve the generation of the required small molecule compound structures.

After the first reward and second reward are determined, a processing result is obtained by subjecting the first reward and the second reward to consolidation processing. Based on the processing result, the initial agent is updated to the target agent using a policy gradient algorithm.

In some embodiments, the policy gradient algorithm is a Monte Carlo policy gradient algorithm. In some embodiments, the policy gradient algorithm is used in training with the small molecule compound structures to generate a target agent.

FIG. 2C is a flowchart of yet another embodiment of a process for generating a molecular structure of a chemical compound. In some embodiments, the process 400 is implemented in the computer system 100 of FIG. 1 and comprises:

In some embodiments, the target neural network model is a recurrent neural network model, and operations 410 and 420 correspond with operations 210 and 220 of FIG. 2A, respectively.

Referring back to FIG. 2C, in 430, the computer system determines the small molecule compound structure symbol corresponding to the current step based on the current cell state of at least one step in the recurrent neural network model.

In 440, the computer system combines the small molecule compound structure symbols corresponding to the at least one step in the recurrent neural network model to form the small molecule compound structure sequence. In some embodiments, the small molecule compound structure sequence is in a simplified molecular-input line-entry system (SMILES) specification.

In some embodiments, the target neural network model is a recurrent neural network (RNN) model. The RNN model is treated as a partially observable Markov decision process. In process 400, the RNN model determines the action (i.e., the small molecule compound structure (SMILES) symbol) corresponding to the current step based on the current cell state of each step. Subsequently, the small molecule compound structure sequence is formed by combining the small molecule compound structure symbols corresponding to at least one step in the recurrent neural network model. For example, the small molecule compound structure symbols corresponding to each step a₁, a₂ . . . a_{T} are combined to form the small molecule compound structure sequence A, i.e., the small molecule compound structure sequence A=a₁, a₂ . . . a_{T}.

FIG. 2D is a flowchart of yet another embodiment of a process for generating a molecular structure of a chemical compound. In some embodiments, the process 500 is implemented in the computer system 100 of FIG. 1 and comprises:

In some embodiments, the target neural network model is a recurrent neural network (RNN) model, and operations 510 and 520 correspond with operations 210 and 220 of FIG. 2A, respectively.

Referring back to FIG. 2D, in 530, the computer system acquires a small molecule compound structure sequence set.

In 540, the computer system acquires a vocabulary corresponding to each small molecule compound structure sequence of the at least one small molecule compound structure sequence. A vocabulary can be formatted in a <key, value> structure. As an example of the vocabulary, one “small molecule compound structure sequence” corresponds to one number.

In 550, the computer system adds a first token and a second token to each small molecule compound structure sequence, and adds the first token and the second token to the vocabulary corresponding to each small molecule compound structure sequence.

In some embodiments, the small molecule compound structure sequence set comprises: at least one small molecule compound structure sequence.

In some embodiments, because the first token indicates the start position and the second token indicates the end position, the first token is represented by a GO token, and the second token is represented by an END token.

Optionally, the process 500 further comprises:

In 560, the computer system pretrains an initial neural network model based on the small molecule compound structure sequence set to obtain the target neural network model.

In some embodiments, the small molecule compound structure sequence set is acquired and pretreated, and, for each small molecule compound structure sequence, a corresponding vocabulary is acquired. As an example, the pretreating of the small molecule compound structure sequence set includes acquiring a vocabulary and adding tokens to the small molecule compound structure sequence set. For example, a GO token is added at the start position of each small molecule compound structure sequence, and an END token is added at the end position of each small molecule compound structure sequence to facilitate the use of the small molecule compound structure sequence set in subsequent pretraining of the RNN model and to guide the RNN model start position and end position towards generation of a target molecular structure of the chemical compound. The GO and END tokens are also added to the vocabularies.

In some embodiments, the pretraining operation described above acquires vocabularies and adds tokens to the beginnings and ends of the small molecule compound structure sequences. In some embodiments, lessons are drawn from the concept of word embedding. In other words, the vocabulary is used to determine an embedding matrix. The embedding matrix can be input in each step of the RNN model, with each small molecule symbol converted into a real-valued vector.

Please note that the embedding matrix can correspond with a two-dimensional matrix, one dimension corresponds with the size of the vocabulary, and the other dimension corresponds with the length of the embedding vector. During the input for each step, the atomic symbol (e.g., chemical element) of that step can be used to look up a one-dimensional vector in the embedding matrix. Equivalently, each atomic symbol can be converted into a vector representation by querying the embedding matrix and the embedding matrix can serve as an RNN model input argument.

FIG. 2E is a flowchart of an embodiment of a process for pretraining an initial neural network model. In some embodiments, the process 600 is an implementation of operation 560 of FIG. 2D and comprises:

In 610, the computer system selects a small molecule compound structure training sequence from the small molecule compound structure sequence set.

In 620, the computer system converts, using a vocabulary corresponding to the small molecule compound structure training sequence, the symbols (corresponding to each step in the initial neural network model) in the small molecule compound structure training sequence into vector representations.

In 630, the computer system sets the first token as the input argument for the initial neural network model, and generates a small molecule compound structure sequence step by step in the initial neural network model.

In 640, the computer system adds up the loss values corresponding to each step in the initial neural network model to obtain a statistical result.

In 650, the computer system updates, based on the statistical result, the initial neural network model to the target neural network model using backpropagation through time.

FIG. 2F is a flowchart of another embodiment of a process for pretraining an initial neural network model. In some embodiments, the process 700 is an implementation of operation 560 of FIG. 2D and comprises:

In 710, the computer system selects a small molecule compound structure training sequence from the small molecule compound structure sequence set, and converts the symbols (corresponding to each step in the initial neural network model) in the small molecule compound structure training sequence into vector representations based on the vocabulary corresponding to the small molecule compound structure training sequence.

In 720, the computer system starts generating a small molecule compound structure sequence step by step in the initial neural network model using the “GO token” as an RNN model input argument.

In 730, the computer system adds up the loss values (log_loss) corresponding to each step in the initial neural network model to calculate a total to obtain a statistical result. In some embodiments, the computer system updates, based on the statistical result, the initial neural network model to the target neural network model using backpropagation through time.

Optionally, the process 700 further comprises:

In 740, the computer system calculates a sampling probability corresponding to each step in the initial neural network model based on a first quantity and a second quantity.

In 750, the computer system conducts, based on the sampling probability corresponding to each step in the initial neural network model, a Bernoulli trial to obtain each corresponding calculation result.

In 750, in the event that the calculation result is a first numerical value, the computer system sets the vector representation converted from the symbol corresponding to the previous step in the small molecule compound structure training sequence as the input argument for the current step.

In 760, in the event that the calculation result is a second numerical value, the computer system sets the output argument of the previous step in the small molecule compound structure training sequence as the input argument of the current step.

In some embodiments, the first quantity is the current number (epoch_num) of iterations in relation to the small molecule compound structure sequence set in the pretraining operation, and the second quantity is the total number (total_epoch) of iterations in relation to the small molecule compound structure sequence set in the pretraining operation.

Optionally, the sampling probability is the Bernoulli sampling parameter p_ber. In some embodiments, the following formula based on the first quantity epoch_num and the second quantity total_epoch is used to calculate the sampling probability corresponding to each step in the initial neural network model.

$$p\_ber = \frac{total\_epoch}{{total}_{epoch} + exp ( \frac{{epoch}_{num}}{{total}_{epoch}} )}$$

The epoch_num can represent the current number of iterations on the small molecule library set, and the total_epoch can represent the total number of iterations for the small molecule library set.

After obtaining the sampling probability corresponding to each step in the initial neural network model, the sampling probability corresponding to each step in the neural network model is used as a Bernoulli distribution trial parameter. A Bernoulli trial is performed to obtain a calculation result corresponding to each step. In the event that the calculation result is 1, the symbol corresponding to the previous step in the small molecule compound structure trial sequence is converted into a vector, i.e., the true vector value (ground truth input), to serve as the input argument for the current step. In the event that the result is 0, the output argument of the previous step is set as the input argument of the current step.

In some embodiments, the calculation technique for performing sampling calculations is used to ensure that, with regard to the input argument of each step of the RNN model in the initial stage of the training process, there is a greater tendency to select the output argument of the previous step to serve as the input argument of the current step, thus ensuring faster convergence of input arguments when training begins. When the training operation is about to end, more possibilities can be explored.

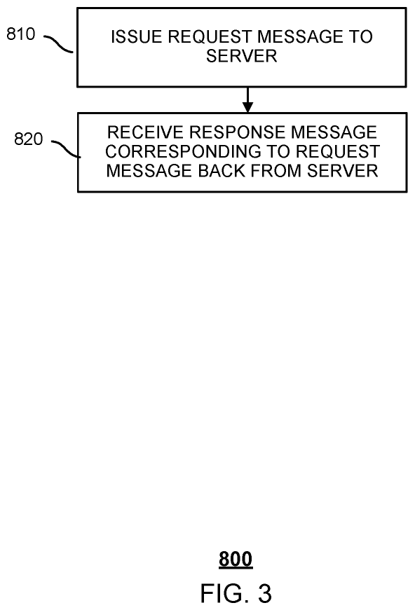

FIG. 3 is a flowchart of yet another embodiment of a process for generating a molecular structure of a chemical compound. In some embodiments, the process 800 is implemented in the computer system 100 of FIG. 1 and comprises:

In 810, the computer system issues a request message to a server. In some embodiments, the request message is used to request a target agent on a server to generate a target molecular structure of a chemical compound, the target agent being obtained through training based on a first reward and a second reward. In some embodiments, the first reward is a reward determined by the model likelihood of a target neural network model, the second reward is a reward self-defined based on target requirements, and the target agent is configured to determine a molecular compound structure.

In 820, the computer system receives a response message corresponding to the request message back from the server. In some embodiments, the information included in the response message comprises: the target molecular structure of the chemical compound.

In some embodiments, a client issues a request message to a server. In some embodiments, the request message requests a target agent on the server to generate a target molecular structure of the chemical compound. In some embodiments, the target agent is obtained through training based on the first reward and the second reward. The first reward can be a reward determined by the model likelihood of a target neural network model, the second reward can be a reward self-defined based on target requirements, and the target agent can determine a molecular compound structure. A response message corresponding to the request message is received back from the server. In some embodiments, the information included in the response message comprises: the target molecular structure of the chemical compound.

Because a scheduled sampling technique is adopted in the pretraining process, and the target agent is trained based on a first reward and a second reward, a better fit exists between the target agent-based technique of generating small molecule compound structures and the pretraining operation. Because the first reward can be a reward determined by the model likelihood of the target neural network model, and the second reward can be a reward self-defined based on target requirements, the generated small molecule compound structure can meet the specific expectations of the user regarding generation of small molecule compound structures, and the small molecule compound structure generating process becomes partially controllable. Moreover, enabling the target agent to “make the most of itself” while imitating existing distributions is possible and thus a greater number of novel small molecule compound structures can be generated.

Therefore, in some embodiments, a better fit exists between the process of target agent-based generation of small molecule compound structures and the pretraining operation, and expected small molecule compound structures generation is achieved. The technical results of increased small molecule compound structure generation efficiency and controllability are thus realized. The conventional technical issues, namely, the poor fit between the process of agent generation of sequences and the pretraining operation, and the inability to generate small molecule compound structures that meet expectations, are resolved.

Optionally, the process 800 for generating the molecular structure of the chemical compound can be understood as a process for generating small molecule drug structures that is customizable and based on scheduled sampling. Unlike conventional generation processes, embodiments of the present application introduce scheduled sampling in the pretraining operation. In other words, the target agent is trained based on a first reward and a second reward, which increases the fit between the process for target agent-based generation of small molecule compound structures and the pretraining operation, while reducing the limitations of exposure bias.

In some embodiments, the executing entity for the above operations 810 and 820 is an SaaS client. With the process 800 generating a molecular structure of a chemical compound as provided by an embodiment of the present application, there already exists small molecule compound molecular structure sets for input in actual application contexts. The output is the novel small molecule compound structure with the physical and chemical properties expected by the user. This process 800 can be implemented as a small molecule drug design service provided for the user, i.e., customized generation of a novel molecular structure having the features or nature expected by the user.

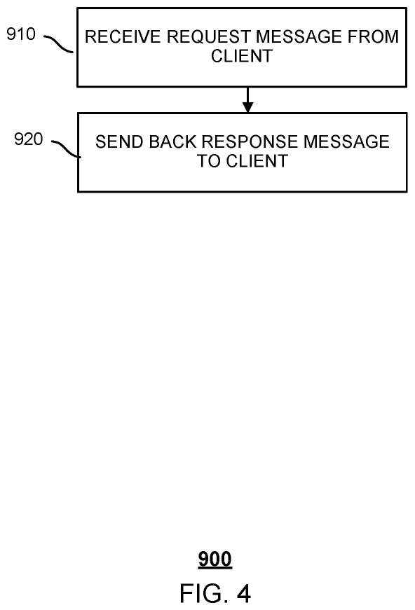

FIG. 4 is a flowchart of yet another embodiment of a process for generating a molecular structure of a chemical compound. In some embodiments, the process 900 is implemented in the computer system 100 of FIG. 1 and comprises:

In 910, the computer system receives a request message from a client. In some embodiments, the request message is used to request a local target agent on a server to generate a target molecular structure of the compound, the target agent being obtained through training based on a first reward and a second reward. In some embodiments, the first reward is a reward determined by the model likelihood of a target neural network model, the second reward is a reward self-defined based on target requirements, and the target agent is configured to determine a molecular compound structure.

In 920, in response to the request message, the computer system sends back a response message to the client. In some embodiments, the information included in the response message comprises: the target molecular structure of the compound.

In some embodiments, a server receives a request message from a client, the request message being used to request a local target agent on a server to generate a target molecular structure of a compound. The target agent can be obtained through training based on a first reward and a second reward. The first reward can be a reward determined by the model likelihood of a target neural network model, the second reward can be a reward self-defined based on target requirements, and the target agent can be used to determine a molecular compound structure. In some embodiments, in response to the request message, a response message can be sent back to the client, and the information included in the response message includes: the target molecular structure of the compound.

Because a scheduled sampling technique is adopted in the pretraining operation, and the target agent is trained based on a first reward and a second reward, a better fit exists between the target agent-based process for generating small molecule compound structures and the pretraining operation. Because the first reward is a reward determined by the model likelihood of the target neural network model, and the second reward is a reward self-defined based on target requirements, the generated small molecule compound structure can meet the specific expectation of the user regarding generation of small molecule compound structures, and the small molecule compound structure generating process can become partially controllable. Moreover, enabling the target agent to “make the most of itself” while imitating existing distributions is possible and thus a greater number of novel small molecule compound structures can be generated.

Therefore, in some embodiments, a better fit exists between the process for target agent-based generation of small molecule compound structures and the pretraining operation, and the generating of the expected small molecule compound structures can be achieved. The technical results of increased small molecule compound structure generation efficiency and controllability thus occurs. The limitations of conventional processes, namely, the poor fit between the process of agent generation of sequences and the pretraining process, and the inability to generate small molecule compound structures that meet expectations, can be reduced.

Optionally, the process 900 for generating a molecular structure of a chemical compound can be understood as a process for generating small molecule drug structures that is customizable and based on scheduled sampling. Unlike conventional generation processes, process 900 introduces scheduled sampling in the pretraining operation. In other words, the target agent is trained based on a first reward and a second reward, which increases the fit between the process for target agent-based generation of small molecule compound structures and the pretraining operation, while reducing exposure bias.

In some embodiments, the executing entity for operations 910 and 920 is an SaaS server. With the process 900 for generating a molecular structure of a chemical compound, small molecule compound molecular structure sets for input in actual application contexts already exist. The output can be the novel small molecule compound structure with the physical and chemical properties expected by the user. This process 900 can be implemented as a small molecule drug design service provided for the user, i.e., customized generation of a novel molecular structure having the features or nature expected by the user.

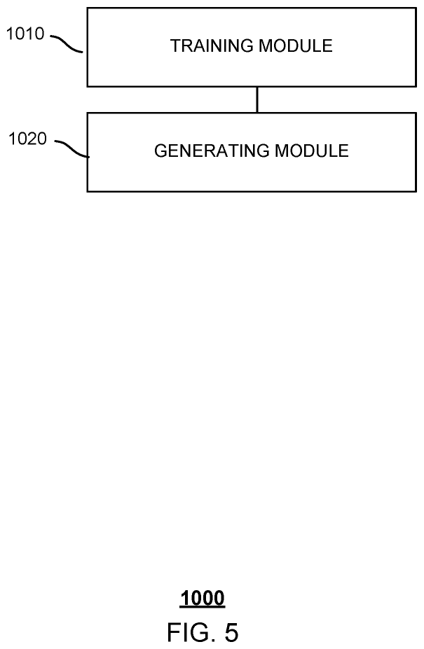

FIG. 5 is a structural diagram of an embodiment of a system for generating a molecular structure of a chemical compound. In some embodiments, the system 1000 is configured to implement process 200 of FIG. 2A and comprises: a training module 1010 and a generating module 1020.

In some embodiments, the training module 1010 is configured to train a target agent based on a first reward and a second reward. In some embodiments, the first reward is a reward determined by the model likelihood of a target neural network model, the second reward is a reward self-defined based on target requirements, and the target agent is configured to determine a molecular compound structure.

In some embodiments, the generating module 1020 is configured to generate a target molecular structure of the compound using the target agent.

The modules described above can be implemented as software components executing on one or more general purpose processors, as hardware such as programmable logic devices and/or Application Specific Integrated Circuits designed to perform certain functions or a combination thereof. In some embodiments, the modules can be embodied by a form of software products which can be stored in a nonvolatile storage medium (such as optical disk, flash storage device, mobile hard disk, etc.), including a number of instructions for making a computer device (such as personal computers, servers, network equipment, etc.) implement the methods described in the embodiments of the present invention. The modules may be implemented on a single device or distributed across multiple devices. The functions of the modules may be merged into one another or further split into multiple sub-modules.

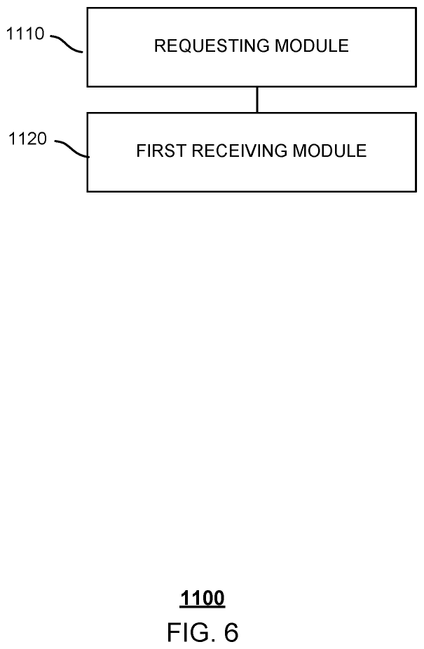

FIG. 6 is a structural diagram of another embodiment of a system for generating a molecular structure of a chemical compound. In some embodiments, the system 1100 is configured to implement process 800 of FIG. 3 and comprises: a requesting module 1110 and a first receiving module 1120.

In some embodiments, the requesting module 1110 is configured to request that a request message be issued to a server. In some embodiments, the request message is used to request a target agent on the server to generate a target molecular structure of the chemical compound. The target agent can be obtained through training based on a first reward and a second reward. The first reward is a reward determined by the model likelihood of a target neural network model, the second reward is a reward self-defined according to target requirements, and the target agent is configured to determine a molecular compound structure.

In some embodiments, the first receiving module 1120 is configured to receive a response message corresponding to the request message back from the server. In some embodiments, the information included in the response message comprises: the target molecular structure of the chemical compound.

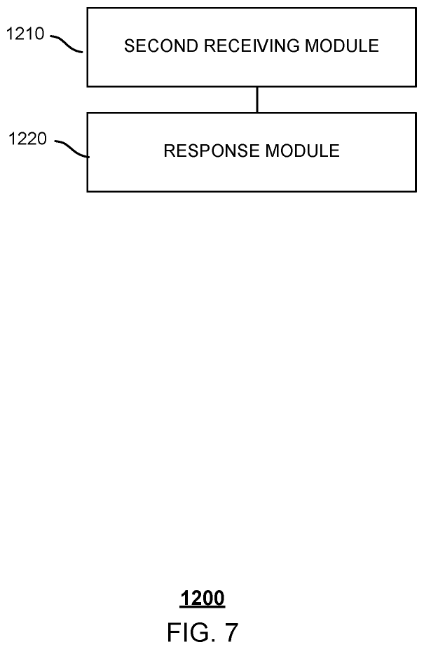

FIG. 7 is a structural diagram of yet another embodiment of a system for generating a molecular structure of a chemical compound. In some embodiments, the system 1200 is configured to implement process 900 of FIG. 4 and comprises: a second receiving module 1210 and a response module 1220.

In some embodiments, the second receiving module 1210 is configured to receive a request message from a client. The request message is used to request a local target agent on the server to generate a target molecular structure of the chemical compound. The target agent is obtained through training based on a first reward and a second reward. In some embodiments, the first reward is a reward determined by the model likelihood of a target neural network model, the second reward is a reward self-defined based on target requirements, and the target agent is configured to determine a molecular compound structure.

In some embodiments, the response module 1220 is configured to send back a response message to the client in response to the request message. In some embodiments, the information included by the response message comprises: the target molecular structure of the chemical compound.

In some embodiments, a system/method/computer program product system includes issuing a request message to a server, wherein the request message requests a target agent on the server to generate a target molecular structure of a compound, the target agent being obtained through training based on a first reward and a second reward, wherein the first reward is a reward determined by a model likelihood of a target neural network model, the second reward is a reward self-defined based on target requirements, and the target agent is configured to determine a molecular compound structure; and receiving a response message corresponding to the request message back from the server, wherein information included in the response message comprises: the target molecular structure of the compound.

In some embodiments, a system/method/computer program product includes receiving a request message from a client, wherein the request message requests a local target agent on a server to generate a target molecular structure of a compound, the target agent is obtained through training based on a first reward and a second reward, the first reward is a reward determined by a model likelihood of a target neural network model, the second reward is a reward self-defined based on target requirements, and the target agent is configured to determine a molecular compound structure; and in response to the receiving of the request message, sending back a response message to the client, wherein information included in the response message comprises: the target molecular structure of the compound.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method, comprising:
training a target agent based on a first reward and a second reward, wherein the first reward is a reward determined by a model likelihood of a target neural network model, the second reward is a reward self-defined based on target requirements, and the target agent is configured to determine a molecular compound structure; and
generating a target molecular structure of a chemical compound using the target agent.

2. The method as described in claim 1, wherein the training on the target agent based on the first reward and the second reward comprises:
acquiring an initial agent;
determining a model likelihood of a small molecule compound structure sequence, generated by the initial agent, in relation to the target neural network model as the first reward and determining a molecular structure limiting conditions set based on the target requirements as the second reward;
subjecting the first reward and the second reward to consolidation processing to obtain a processing result; and
updating, based on the processing result, the initial agent to the target agent using a policy gradient algorithm.

3. The method as described in claim 2, wherein the target neural network model is a recurrent neural network model, and the method further comprises:
determining a small molecule compound structure symbol corresponding to the current step based on a current cell state of at least one step in the recurrent neural network model; and
combining small molecule compound structure symbols corresponding to at least one step in the recurrent neural network model to form the small molecule compound structure sequence.

4. The method as described in claim 1, further comprising:
acquiring a small molecule compound structure sequence set, wherein the small molecule compound structure sequence set comprises: at least one small molecule compound structure sequence;
acquiring a vocabulary corresponding to each small molecule compound structure sequence of the at least one small molecule compound structure sequence; and
adding a first token and a second token to each small molecule compound structure sequence and adding the first token and the second token to the vocabulary corresponding to each small molecule compound structure sequence, wherein the first token is used to indicate a start position, and the second token is used to indicate an end position.

5. The method as described in claim 4, further comprising:
pretraining, based on the small molecule compound structure sequence set, an initial neural network model to obtain a target neural network model.

6. The method as described in claim 5, wherein the pretraining of the initial neural network model to obtain the target neural network model comprises:
selecting a small molecule compound structure training sequence from the small molecule compound structure sequence set;
converting, based on the vocabulary corresponding to each small molecule compound structure sequence of the at least one small molecule compound structure sequence, symbols corresponding to each step in the initial neural network model into vector representations;
setting the first token as an input argument for the initial neural network model to generate a small molecule compound structure sequence step by step in the initial neural network model;
adding up loss values corresponding to each step in the initial neural network model to obtain a statistical result; and
updating, based on the statistical result, the initial neural network model to the target neural network model using backpropagation through time.

7. The method as described in claim 6, further comprising:
calculating a sampling probability corresponding to each step in the initial neural network model based on a first quantity and a second quantity, wherein the first quantity is the current number of iterations in relation to the small molecule compound structure sequence set in the pretraining operation, and the second quantity is the total number of iterations in relation to the small molecule compound structure sequence set in the pretraining operation;
conducting, based on the sampling probability corresponding to each step in the initial neural network model, a Bernoulli trial to obtain each corresponding calculation result;
in the event that a calculation result is a first numerical value, setting a vector representation converted from a symbol corresponding to a previous step in the small molecule compound structure training sequence as an input argument for a current step; and
in the event that a calculation result is a second numerical value, setting an output argument of the previous step as an input argument of the current step.

8. A system, comprising:
a processor; and
a memory coupled with the processor, wherein the memory is configured to provide the processor with instructions which when executed cause the processor to:
train a target agent based on a first reward and a second reward, wherein the first reward is a reward determined by a model likelihood of a target neural network model, the second reward is a reward self-defined based on target requirements, and the target agent is configured to determine a molecular compound structure; and
generate a target molecular structure of a chemical compound using the target agent.

9. A computer program product being embodied in a tangible non-transitory computer readable storage medium and comprising computer instructions for:
training a target agent based on a first reward and a second reward, wherein the first reward is a reward determined by a model likelihood of a target neural network model, the second reward is a reward self-defined based on target requirements, and the target agent is configured to determine a molecular compound structure; and
generating a target molecular structure of a chemical compound using the target agent.
